(19) United States
(12) Patent Application Publication
(10) Pub. No.: US 2010/0267100 A1
(43) Pub. Date: Oct. 21, 2010

(54) Enzymatic synthesis of optically pure beta-hydroxy carboxylic acids and their derivatives

(76) Inventors: Ling Hua, Dallas, TX (US); Dunming Zhu, Dallas, TX (US); Chandrani Mukherjee, Chandernagore (IN); Edward R. Biehl, Dallas, TX (US)

Correspondence Address: BAKER BOTTS L.L.P., 2001 ROSS AVENUE, SUITE 600, DALLAS, TX, 75201-2980, US

(21) Appl. No.: 11/689,243

(22) Filed: Mar. 21, 2007

(51) Int. Cl. C12P 7/40

(52) U.S. Cl. 435/136

(57) ABSTRACT

The present disclosure is related to systems, compositions, and methods for producing a β-hydroxy carboxylic acid and/or a β-hydroxy carboxylic amide in enantiomeric excess (e.g., enantiomerically pure). In some specific example embodiments, a method may include contacting a substrate and/or intermediate with a cyanide, a ketoreductase, a nitrilase, and/or a nitrile hydratase. Systems, compositions, and methods, in some embodiments, may produce a β-hydroxy carboxylic acid and/or a β-hydroxy carboxylic amide in enantiomeric excess under convenient conditions (e.g., pH 5-9 and temperatures under 50° C.).

### TECHNICAL FIELD

The present disclosure is related to systems, compositions, and methods for producing a β-hydroxy carboxylic acid and/or a β-hydroxy carboxylic amide in enantiomeric excess (e.g., enantiomerically pure).

### BACKGROUND OF THE INVENTION

Optically pure β-hydroxy carboxylic acids and their derivatives may be precursors of β-blockers and 1,3-amino alcohols, which may be important intermediates for the synthesis of natural products, antibiotics and chiral auxiliaries. In addition, chiral β-hydroxy carboxylic acids may be used to prepare copolyesters for film, fiber, molding and coating applications. The utility of chiral β-hydroxy carboxylate compounds as useful synthons has stimulated the development of new methodologies for their construction, and a variety of methods have been reported. These include asymmetric acetate aldol reactions, Reformatsky reactions, borane reductions, and lipase-catalyzed resolution of racemic β-hydroxy carboxylic acid esters. However, acetate aldol reactions and Reformatsky reactions may be inconvenient and/or impractical due to the extreme temperatures (e.g., −78° C.) required. Borane reductions are encumbered by the need to use hazardous materials (e.g., borane). Lipase-catalyzed enantiomer resolution may be inefficient with yields of less than 50%.

### SUMMARY OF THE INVENTION

Therefore a need has arisen for methods and compositions for producing β-hydroxy carboxylic acids and/or β-hydroxy carboxylic amides in higher enantiomeric excess, under more convenient conditions, and/or using more environmentally benign materials.

The present disclosure is related to systems, compositions, and methods for producing a β-hydroxy carboxylic acid and/or a β-hydroxy carboxylic amide in enantiomeric excess (e.g., enantiomerically pure). For example, according to some embodiments of the disclosure, a method of forming a composition comprising an enantiomeric excess of a β-hydroxy carboxylic acid may include contacting an α-haloketone with sodium cyanide in a composition to produce a β-ketonitrile, contacting the β-ketonitrile with one or more ketoreductases under conditions that permit reduction of the β-ketonitrile to form a β-hydroxynitrile, and contacting the β-hydroxynitrile with one or more nitrilases under conditions that permit hydrolysis of the β-hydroxynitrile to form an enantiomeric excess of an enantiomer of a β-hydroxy carboxylic acid in the composition.

In some embodiments, a method of forming a composition comprising an enantiomeric excess of a β-hydroxy carboxylic amide may include contacting an α-haloketone with sodium cyanide in a composition to produce a β-ketonitrile, contacting the β-ketonitrile with one or more ketoreductases under conditions that permit reduction of the β-ketonitrile to form a β-hydroxynitrile, and contacting the β-hydroxynitrile with one or more nitrile hydratases under conditions that permit hydrolysis of the β-hydroxynitrile to form an enantiomeric excess of an enantiomer of a β-hydroxy carboxylic amide in the composition.

A method of forming a composition comprising an enantiomeric excess of a β-hydroxy carboxylic acid may include, according to some embodiments, contacting an α-haloketone with one or more ketoreductases under conditions that permit reduction of the α-haloketone to produce an α-haloalcohol, contacting the α-haloalcohol with sodium cyanide to form a β-hydroxynitrile, and contacting the β-hydroxynitrile with one or more nitrilases under conditions that permit hydrolysis of the β-hydroxynitrile to form an enantiomeric excess of an enantiomer of a β-hydroxy carboxylic acid in the composition.

In some embodiments, a method of forming a composition comprising an enantiomeric excess of a β-hydroxy carboxylic amide may include contacting an α-haloketone with one or more ketoreductases under conditions that permit reduction of the α-haloketone to produce an α-haloalcohol, contacting the α-haloalcohol with sodium cyanide to form β-hydroxynitrile, and contacting the β-hydroxynitrile with one or more nitrile hydratases under conditions that permit hydrolysis of the β-hydroxynitrile to form an enantiomeric excess of an enantiomer of a β-hydroxy carboxylic amide in the composition.

A method of forming a composition comprising an enantiomeric excess of a β-hydroxy carboxylic acid may include, according to some embodiments, contacting an α-haloketone with sodium cyanide to produce a β-ketonitrile, contacting the β-ketonitrile with one or more nitrilases under conditions that permit hydrolysis of the β-ketonitrile to form at least one β-keto acid, and contacting the β-keto acid with a ketoreductase to form an enantiomeric excess of an enantiomer of a β-hydroxy carboxylic acid in the composition.

According to some embodiments, a method of forming a composition comprising an enantiomeric excess of a β-hydroxy carboxylic amide may include contacting an α-haloketone with sodium cyanide to produce a β-ketonitrile, contacting the β-ketonitrile with one or more nitrile hydratases under conditions that permit hydrolysis of the β-ketonitrile to form at least one β-keto amide, and contacting the β-keto amide with a ketoreductase to form to form an enantiomeric excess of an enantiomer of a β-hydroxy carboxylic amide in the composition.

A method of forming a composition comprising an enantiomeric excess of a β-hydroxy carboxylic acid and/or a β-hydroxy carboxylic amide, in some embodiments, may yield an excess of one enantiomer over another. For example, the enantiomeric excess may be over about 80 mole percent, over about 95 mole percent, or over about 99 mole percent.

In some embodiments, a ketoreductase, a nitriliase, and/or a nitrile hydratase may contact a substrate and/or intermediate under a wide range of temperature and pH. For example, conditions that permit hydrolysis of a β-hydroxynitrile (e.g., by a nitrilase and/or a nitrile hydratase) may include a pH of between about 4 and about 10 and a temperature of less than about 60° C.

A composition may include water and/or an organic solvent, in some embodiments. An α-haloketone may include, for example, a molecule with the formula R—C(O)—CH₂—X, wherein R may include any organic substituent (e.g., alkyl, alkenyl, alkynyl, cycloalkyl, cycloalkenyl, cycloalkynyl, and aryl, each of which may have one or more carbon and/or hydrogen atoms replaced by S, O, N, P) and X is a halogen (e.g., chlorine, bromine, fluorine, and iodine). For example, an α-haloketone may include a molecule selected from the group consisting of:

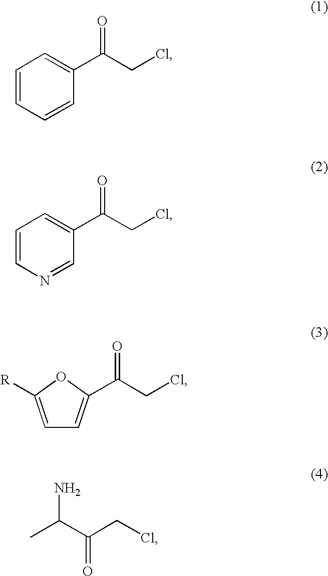

and/or variants (e.g., brominated variants) thereof. In some embodiments, a β-hydroxy carboxylic acid may include a molecule selected from the group consisting of:

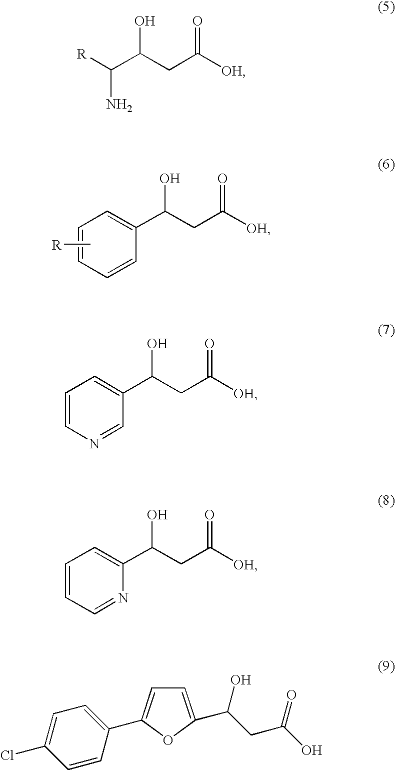

and/or variants thereof.

Conditions that permit reduction by a ketoreductase may include contacting the substrate and/or ketoreductase with a cofactor (e.g., an oxidized and/or reduced form of NADH and/or NADPH). Conditions that permit reduction by a ketoreductase may include contacting a cofactor with glucose and glucose dehydrogenase and/or formate and formate dehydrogenase. Additional cofactor regeneration systems may include, for example, self-regeneration, phosphate dehydrogenase regeneration (e.g., with glucose-6-phosphase), hydrogenase-regeneration (e.g., with molecular hydrogen), and/or alcohol dehydrogenase regeneration (e.g., with ethanol).

Systems, compositions, and methods, according to some embodiments of the disclosure, may include one or more buffers, salts, surfactants, and/or other reagents. In addition, systems, compositions, and methods may include one or more reaction vessels the physically and/or or chemically separate one or more substrates, intermediates, products, enzymes, catalysts, oxidizing agents, reducing agents, cofactors, coenzymes, and/or other reagents.

### BRIEF DESCRIPTION OF THE DRAWINGS

A more complete and thorough understanding of the present embodiments and advantages thereof may be acquired by referring to the following description taken in conjunction with the accompanying drawings, in which like reference numbers indicate like features, and wherein:

FIG. 1 is a scheme illustrating an example embodiment of a process for forming a β-hydroxy carboxylic acid and/or a β-hydroxy carboxylic amide;

FIG. 2 is a scheme illustrating an example embodiment of a process for forming a β-hydroxy carboxylic acid and/or a β-hydroxy carboxylic amide; and

FIG. 3 is a scheme illustrating an example embodiment of a process for forming a β-hydroxy carboxylic acid and/or a β-hydroxy carboxylic amide.

### DETAILED DESCRIPTION OF THE INVENTION

The present disclosure relates to methods, compositions, and systems for producing a β-hydroxy carboxylic acid and/or a β-hydroxy carboxylic amide.

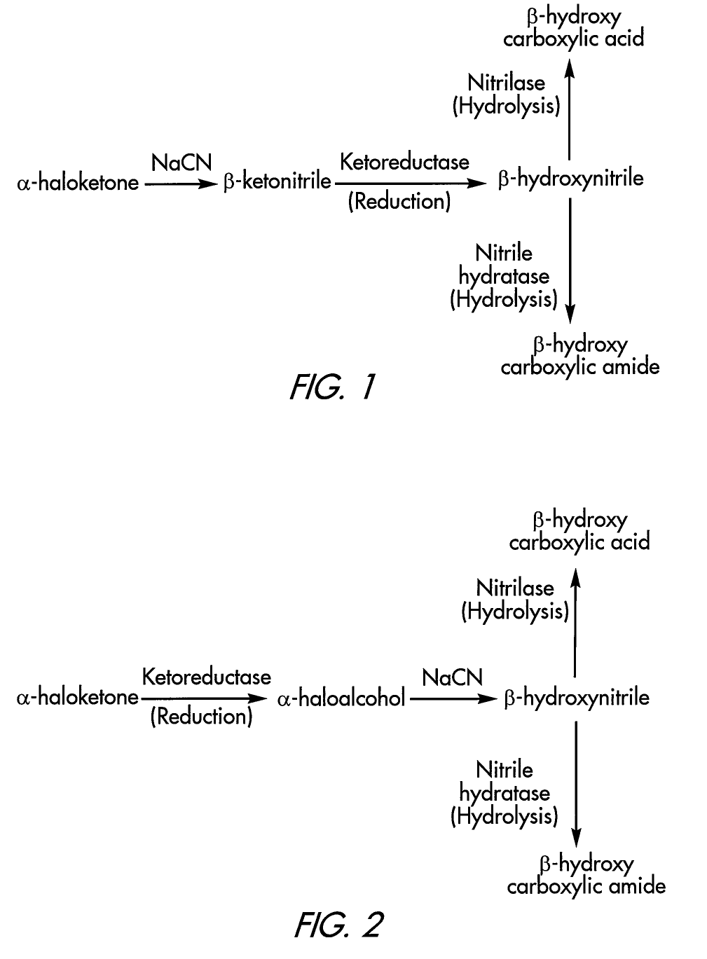

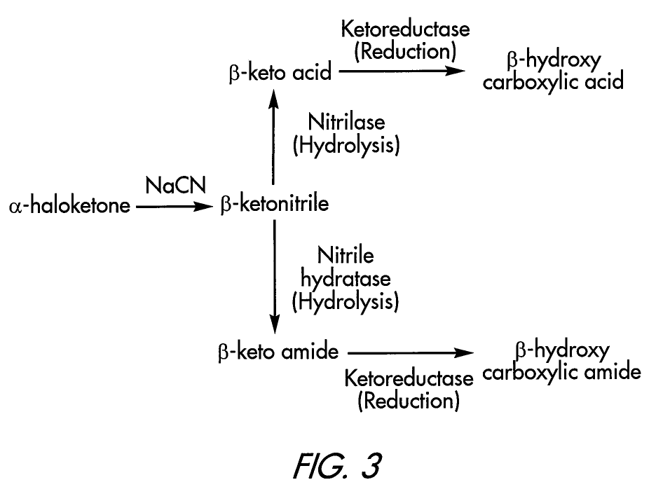

A method for producing a β-hydroxy carboxylic acid and/or a β-hydroxy carboxylic amide may include, in some embodiments, contacting an α-haloketone with a ketoreductase to form an α-haloalcohol (e.g., FIG. 2). In some embodiments, a method for producing a β-hydroxy carboxylic acid and/or a β-hydroxy carboxylic amide may include contacting a β-ketonitrile with a ketoreductase to form a β-hydroxynitrile (e.g., FIG. 1). A method for producing a β-hydroxy carboxylic acid may include contacting a β-keto acid with a ketoreductase to form a β-hydroxy carboxylic acid, in some embodiments (e.g., FIG. 3). A method for producing a β-hydroxy carboxylic amide may include, in some embodiments, contacting a β-keto amide with a ketoreductase to form a β-hydroxy carboxylic amide (e.g., FIG. 3). In some embodiments, a method for producing a β-hydroxy carboxylic acid may include contacting a β-hydroxynitrile with a nitrilase to form a β-hydroxy carboxylic acid (e.g., FIGS. 1 & 2). A method for producing a β-hydroxy carboxylic amide may include contacting a β-hydroxynitrile with a nitrile hydratase to form a β-hydroxy carboxylic amide, in some embodiments (e.g., FIGS. 1 & 2). A method for producing a β-hydroxy carboxylic acid and/or a β-hydroxy carboxylic amide may include, in some embodiments, contacting a β-ketonitrile with a nitrilase to form a β-keto acid (e.g., FIG. 3). In some embodiments, a method for producing a β-hydroxy carboxylic acid and/or a β-hydroxy carboxylic amide may include contacting a β-ketonitrile with a nitrile hydratase to form a β-keto amide (e.g., FIG. 3). A method for producing a β-hydroxy carboxylic acid and/or a β-hydroxy carboxylic amide may include, in some embodiments, contacting an α-haloalcohol with a cyanide compound (e.g., sodium cyanide) to form a β-ketonitrile (e.g., FIG. 2). A method for producing a β-hydroxy carboxylic acid and/or a β-hydroxy carboxylic amide may include, in some embodiments, contacting an α-haloketone with a cyanide compound (e.g., sodium cyanide) to form a β-ketonitrile (e.g., FIGS. 1 & 3).

According to some embodiments, (R) and/or (S) enantiomers of β-hydroxy carboxylic acids may be prepared in high optical purity (e.g., ee≧95%) from α-chloroketones or α-bromoketones via two coupled ketoreductase/nitrilase enzymatic approaches. For example, an α-chloroketone or α-bromoketone may be converted to a β-ketonitrile, which then may be reduced to an (R) or (S) enantiomer of a β-hydroxynitrile in high enantiomeric purity (Scheme 1, infra). In another example, the cyanization/reduction sequence may be reversed. An α-chloroketone or an α-bromoketone may be first reduced to optically pure α-haloalcohol, which then may be converted to the corresponding β-hydroxynitrile by cyanization with sodium cyanide or other cyanide compounds (Scheme 2, infra). The obtained β-hydroxynitriles may be treated with one or more nitrilases to give the corresponding enantiomerically pure β-hydroxy carboxylic acids.

In some embodiments, synthesis of an enantiomerically pure compound (e.g., a drug) may involve enzymes or other catalysts that produce an enantiomeric excess (ee) of the desired drug enantiomer (or its chemical precursor). A composition comprising a β-hydroxy carboxylic acid and/or a β-hydroxy carboxylic amide produced in accordance with some embodiments of the disclosure may have an excess of one enantiomer relative to another enantiomer. For example, a composition may include more than about 70 molar percent of one enantiomer, more than about 80 molar percent of one enantiomer, more than about 85 molar percent of one enantiomer, more than about 90 molar percent of one enantiomer, more than about 95 molar percent of one enantiomer, more than about 98 molar percent of one enantiomer, or more than about 99 molar percent of one enantiomer. A composition may include up to about 95 molar percent of one enantiomer, up to about 98 molar percent of one enantiomer, up to about 99 molar percent of one enantiomer, or up to about 100 molar percent of one enantiomer (i.e., enantiomerically pure).

A method for producing a composition comprising a β-hydroxy carboxylic acid and/or a β-hydroxy carboxylic amide produced in accordance with some embodiments of the disclosure may produce the desired compound in high yield (moles of product over moles of substrate). For example, a method for producing a composition comprising a β-hydroxy carboxylic acid and/or a β-hydroxy carboxylic amide may have a yield of more than about 50 percent, more than about 55 percent, more than about 60 percent, more than about 65 percent, more than about 70 percent, more than about 75 percent, more than about 80 percent, more than about 85 percent, more than about 90 percent, more than about 95 percent, or more than about 98 percent.

In some embodiments, a β-hydroxy carboxylic acid may be obtained in high enantiomeric purity via chemoenzymatic dynamic kinetic resolution of racemic β-hydroxynitriles followed by chemical hydrolysis of nitrile functional group. This scheme may offer an attractive route to chiral β-hydroxy carboxylic acids since racemic β-hydroxynitriles may be readily accessible by the cyanization of α-haloketones with sodium cyanide followed by NaBH₄ reduction or ring-opening of epoxides by sodium cyanide. However, chemical hydrolysis of nitriles to carboxylic acids may require drastic conditions (e.g., strong bases or acids and relatively high reaction temperature), and may produce unwanted byproducts and/or large amounts of inorganic wastes. For example, chemical hydrolysis of nitriles with β-hydroxy group may result in the undesirable elimination of OH, yielding unsaturated by-products.

Conditions that permit conversion of an α-haloketone to a β-hydroxy carboxylic acid and/or a β-hydroxy carboxylic amide (e.g., an overall reaction and/or at least one intermediate step), according to some embodiments, may include a pH of more than about 1, more than about 2, more than about 3, more than about 4, more than about 5, more than about 6, more than about 7, more than about 8, more than about 9, more than about 10, more than about 11, more than about 12, more than about 13, and/or more than about 14. In some embodiments, conditions that permit conversion of an α-haloketone to a β-hydroxy carboxylic acid and/or a β-hydroxy carboxylic amide may include a pH of less than about 1, less than about 2, less than about 3, less than about 4, less than about 5, less than about 6, less than about 7, less than about 8, less than about 9, less than about 10, less than about 11, less than about 12, less than about 13, and/or less than about 14. Conditions that permit conversion of an α-haloketone to a β-hydroxy carboxylic acid and/or a β-hydroxy carboxylic amide, in some embodiments, may include a pH within a range with endpoints defined by any of the foregoing pHs. For example, a pH range may be from about 2 to about 12, from about 2 to about 8, from about 6 to about 12, from about 3 to 11, from about 4 to about 10, and/or from about 5 to about 9.

According to some embodiments, conditions that permit conversion of an α-haloketone to a β-hydroxy carboxylic acid and/or a β-hydroxy carboxylic amide (e.g., an overall reaction and/or at least one intermediate step), may include a temperature of more than about 0° C., more than about 10° C., more than about 20° C., more than about 40° C., more than about 50° C., more than about 60° C., more than about 80° C., and/or more than about 90° C. Conditions that permit conversion of an α-haloketone to a β-hydroxy carboxylic acid and/or a β-hydroxy carboxylic amide, according to some embodiments, may include a temperature of less than about 100° C., less than about 90° C., less than about 80° C., less than about 60° C., less than about 50° C., less than about 40° C., less than about 20° C., and/or less than about 10° C. Conditions that permit conversion of an α-haloketone to a β-hydroxy carboxylic acid and/or a β-hydroxy carboxylic amide, in some embodiments, may include a temperature within a range with endpoints defined by any of the foregoing temperatures. For example, a temperature range may be from about 0° C. to about 100° C., from about 0° C. to about 50° C., from about 50° C. to about 100° C., from about 10° C. to about 90° C., from about 10° C. to about 60° C., and/or 10° C. to about 50° C.

According to some embodiments of the disclosure, however, the need for drastic conditions (e.g., required by some chemical methods) may be reduced or eliminated. For example, biocatalytic hydrolysis of a nitrile may be performed at an approximately neutral pH (e.g., about 7.0) and/or at approximately room temperature (e.g., about 20° C.). A nitrile may be biotransformed to a carboxylic acid at a pH from about 5 to about 9 and/or a temperature below about 60° C. A nitrile may be biotransformed to a carboxylic acid at a pH from about 6 to about 8 and/or a temperature from about 10° C. to about 50° C. A nitrile may be biotransformed to a carboxylic acid at a pH from about 6.5 to about 7.5 and/or a temperature from about 30° C. to about 50° C. In some example embodiments, a nitrilase may hydrolyze an aromatic β-hydroxy nitrile to a β-hydroxy carboxylic acid at about pH 7.2 and at about 30° C. In some example embodiments, a nitrilase may hydrolyze a β-hydroxy nitrile to a β-hydroxy carboxylic acid at about pH 7.2 and at about 30° C. In some example embodiments, a nitrilase may hydrolyze α,ω-dinitriles to ω-cyanocarboxylic acids at about pH 7.2 and at about 30° C. A process of forming an enantiomerically enriched β-hydroxy carboxylic acid, in some embodiments, may include hydrolyzing a nitrile having a labile functional group (e.g., a functional group that cannot tolerate harsh conditions) without modifying or degrading the labile functional group.

A process of forming an enantiomerically enriched β-hydroxy carboxylic acid, in some embodiments, may include contacting a substrate (e.g., ketones with various structures) with a ketoreductase (e.g., a recombinant ketoreductase). For example, a process of forming an enantiomerically enriched β-hydroxy carboxylic acid (e.g., optically pure) may include a sequential enzymatic approach using ketoreductases and nitrilases. β-ketonitriles may be reduced to enantiomerically pure (R)- or (S)-β-hydroxynitriles with ketoreductases. These (R)- or (S)-β-hydroxynitriles may be hydrolyzed by a nitrilase to form (R)- or (S)-β-hydroxy carboxylic acids, respectively, as shown in Scheme 1. The P-ketonitriles may be prepared by the cyanization of α-haloketones with sodium cyanide.

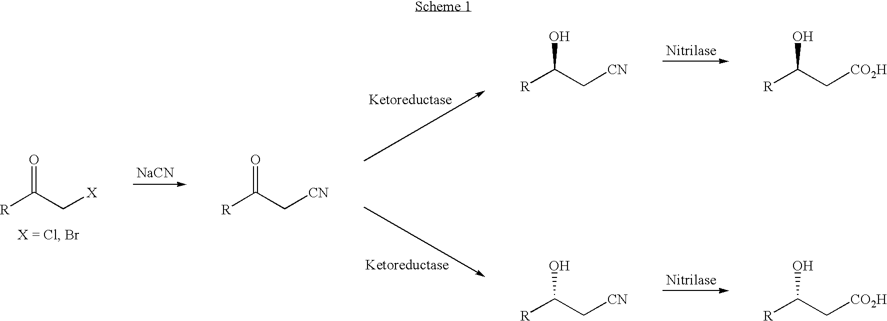

Alternatively, α-chloroketones may be first reduced with ketoreductases. The resulting optically pure α-chloroalcohols were treated with sodium cyanide to give (R)- or (S)-β-hydroxynitriles, which were then hydrolyzed with nitrilase to afford (R)- or (S)-β-hydroxy carboxylic acids (Scheme 2).

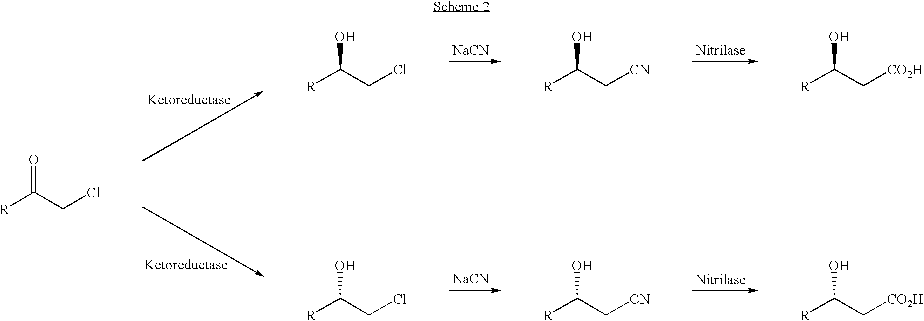

In some embodiments, β-ketonitriles may be reduced with ketoreductases (KRED) and a cofactor. A cofactor may include nicotinamide adenine dinucleotide (NADH) and/or nicotinamide adenine dinucleotide phosphate (NADPH). A cofactor (e.g., NADH or NADPH) may be regenerated with D-glucose and D-glucose dehydrogenase (GDH) systems or formate and formate dehydrogenase (FDH) systems, as shown in Scheme 3 and 4.

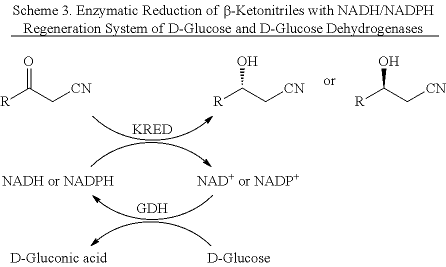

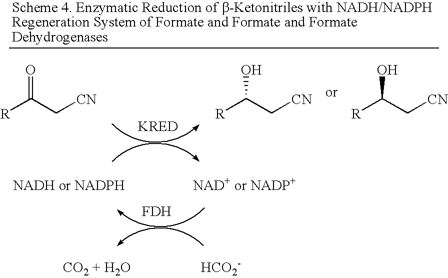

A ketoreductase may reduce a ketone to a chiral alcohol according to some embodiments of the disclosure. A ketoreductase may comprise an enzyme and may be comprised in a composition and/or a whole cell (e.g., a microorganism). For example, a β-ketonitrile may be reduced with a carbonyl reductase from Candida magnoliae (CMCR) to a (R)-β-hydroxynitrile in high yield. The results from an example experiment, Example 1 below, are summarized in Table 1.

TABLE 1
Reduction of β-Ketonitriles Catalyzed by the
Carbonyl Reductases

| R | Ketoreductase | Yield (%) | Ee (%) |
|---|---|---|---|
| Phenyl | CMCR | 92 | 99 (R) |
| | KRED116 | 84 | 99 (S) |
| 4-Fluorophenyl | CMCR | 79 | 99 (R) |
| | KRED130 | 93 | 99 (S) |
| 4-Chlorophenyl | CMCR | 81 | 99 (R) |
| | KRED130 | 86 | 99 (S) |
| 4-Bromophenyl | CMCR | 84 | 99 (R) |
| | KRED130 | 80 | 99 (S) |
| 4-Methylphenyl | CMCR | 69 | 99 (R) |
| | KRED130 | 87 | 99 (S) |
| tert-Butyl | KRED113 | 89 | 99 (R) |
| | KRED132 | 90 | 99 (S) |

As shown in Table 1, a commercially available ketoreductase, KRED113, catalyzed the reduction of aliphatic β-ketonitriles such as 4,4-dimethyl-3-oxo-pentanonitrile to the enantiomerically pure (R)-β-hydroxynitriles in high yields. As also shown in Table 1, (S)-enantiomers of β-hydroxynitriles may be prepared in high yields by reduction of the corresponding β-ketonitriles using other commercially available ketoreductases, such as KRED116, 130 and 132.

Enantiomerically pure (R) and (S)-β-hydroxynitriles were then hydrolyzed to (R) and (S)-β-hydroxy carboxylic acids in high yields, respectively using either nitrilase b116402 from Bradyrhizobium japonicum strain USDA110 or nitrilase from cyanobacterium Synechocystis sp. strain PCC 6803. Results from an example experiment, Example 4, are presented in Table 2.

TABLE 2
Nitrilase Catalyzed Hydrolysis of β-Hydroxynitriles

| R | Enantiomer | Yield (%) | Ee (%) |
|---|---|---|---|
| Phenyl | R | 62 | 99 |
| | S | 89 | 99 |
| 4-Fluorophenyl | R | 91 | 99 |
| | S | 94 | 99 |
| 4-Chlorophenyl | R | 75 | 99 |
| | S | 95 | 99 |
| 4-Bromophenyl | R | 85 | 99 |
| | S | 92 | 99 |
| 4-Methylphenyl | R | 64 | 99 |
| | S | 84 | 99 |
| tert-Butyl | R | 82 | 99 |
| | S | 96 | 99 |

Enantiomerically pure (R) and (S)-β-hydroxynitriles may also be prepared by cyanization of the corresponding optically pure (R) and (S)-chloroalcohols (Example 3), which in turn may be obtained by the reduction of the α-chloroketones (Example 2) under the action of ketoreductases (Scheme 2). Tables 3 and 4 list some examples.

TABLE 3
Ketoreductase Catalyzed Reduction of α-Chloroacetophenones

| R | Ketoreductase | Yield (%) | Ee (%) |
|---|---|---|---|
| 3′-Chlorophenyl | KRED112 | 69 | 99 (R) |
| | KRED130 | 99 | 99 (S) |
| 3′,4′- | KRED112 | 79 | 99 (R) |
| dichlorophenyl | KRED130 | 88 | 99 (S) |
| 4′-Nitrophenyl | KRED107 | 87 | 99 (R) |
| | KRED130 | 76 | 99 (S) |

TABLE 4
Preparation of the Enantiomerically Pure (R)
and (S)-β-Hydroxynitriles via Cyanization of α-Chloroalcohols

| R | Enantiomer | Yield (%) | Ee (%) |
|---|---|---|---|
| 3′-Chlorophenyl | R | 72 | 99 |
| | S | 84 | 99 |
| 3′,4′- | R | 63 | 99 |
| dichlorophenyl | S | 66 | 99 |
| 4′-Nitrophenyl | R | 87 | 99 |
| | S | 90 | 99 |

The obtained optically pure (R) and (S)-β-hydroxynitriles were hydrolyzed under the action of the nitrilase b116402 from Bradyrhizobium japonicum (Example 4) to give the corresponding (R) and (S)-β-hydroxy carboxylic acids, respectively. The results are summarized in Table 5.

TABLE 5
Nitrilase Catalyzed Hydrolysis of Optically
Pure β-Hydroxynitriles

| R | Enantiomer | Yield (%) | Ee (%) |
|---|---|---|---|
| 3′-Chlorophenyl | R | 56 | 99 |
| | S | 82 | 99 |
| 3′,4′- | R | 63 | 99 |
| dichlorophenyl | S | 72 | 99 |
| 4′-Nitrophenyl | R | 57 | 99 |
| | S | 73 | 99 |

In some embodiments, a nitrilase cloned and purified from Bradyrhizobium japonicum strain USDA110 may catalyze enantioselective hydrolysis of a β-hydroxy nitrile (e.g., without other enzymes). When used in concert with, for example, a ketoreductase, enantioselectivity and/or yield of a desired product may be improved relative to the nitrilase alone.

The disclosure also relates, in some embodiments, to a nitrilase and catalytically active variants thereof. Specific example embodiments of nitrilases may include nitrilase b116402 from Bradyrhizobium japonicum and nitrilase from cyanobacterium Synechocystis sp. strain PCC 6803. According to some embodiments, a nitrilase may have catalytic activity over a broad range of temperature (e.g., up to about 50° C., up to about 60° C., up to about 70° C., up to about 80° C., up to about 90° C., and/or up to about 100° C.) and/or pH (e.g., from about 1 to about 14, from about 1 to about 9, from about 5 to about 14, from about 4 to about 10, and/or from about 5 to about 9). A nitrilase (e.g., nitrilase b116402) may also have, in some embodiments, catalytic activity in the presence of one or more organic solvents. According to some embodiments, a nitrilase (e.g., nitrilase b116402) may convert α-hydroxynitriles to α-carboxylic acids with a Vₘₐₓ from about 0.001 U/mg to about 1000 U/mg and/or a Kₘ from about 10 μM to about 10 mM. For example, a Vₘₐₓ for this conversion may be from about 0.001 U/mg to about 0.010 U/mg, from about 0.010 U/mg to about 0.100 U/mg, from about 0.100 U/mg to about 1.0 U/mg, from about 1.0 U/mg to about 10.0 U/mg, or from about 10.0 U/mg to about 100.0 U/mg. Similarly, a Kₘ for this conversion may be from about 10 μM to about 100 μM, from about 100 μM to about 1 mM, or from about 1.0 mM to about 10 mM.

According to some embodiments, a nitrilase (e.g., nitrilase b116402) may convert β-hydroxynitriles to β-carboxylic acids with a Vₘₐₓ from about 0.001 U/mg to about 100 U/mg and/or a Kₘ from about 10 μM to about 10 mM. For example, a Vₘₐₓ for this conversion may be from about 0.001 U/mg to about 0.010 U/mg, from about 0.010 U/mg to about 0.100 U/mg, from about 0.100 U/mg to about 1.0 U/mg, from about 1.0 U/mg to about 10.0 U/mg, or from about 10.0 U/mg to about 100.0 U/mg. Similarly, a Kₘ for this conversion may be from about 10 μM to about 100 μM, from about 100 μM to about 1 mM, or from about 1.0 mM to about 10 mM.

A nitrilase, according to some embodiments of the disclosure, may include a polypeptide having the amino acid sequence of SEQ ID NO: 2 and/or variants thereof. For example, a nitrilase capable of hydrolyzing nitriles to carboxylic acids with a Vₘₐₓ of at least about 0.001 U/mg and/or a Kₘ below about 10 mM may include a polypeptide with an amino acid sequence that is more than about 20%, more than about 40%, more than about 60%, more than about 70%, more than about 80%, more than about 85%, more than about 90%, more than about 95%, more than about 97%, more than about 98%, or more than about 99% identical to SEQ ID NO: 2. In some embodiments, a nitrilase may include a polypeptide having an amino acid sequence that is more than about 95% identical to SEQ ID NO: 2 and/or may have a catalytic activity sufficient to convert at least about 50% of a nitrile to a carboxylic acid at a pH from about 5 to about 9 and a temperature less than 60° C.

A nitrilase, according to some embodiments of the disclosure, may be encoded by a nucleic acid (e.g., DNA or RNA) comprising a nucleic acid having the sequence of SEQ ID NO: 1 and/or variants thereof. For example, a nitrilase capable of hydrolyzing nitriles to carboxylic acids with a Vₘₐₓ of at least about 0.001 U/mg and/or a Kₘ below about 10 mM may be encoded by a nucleic acid comprising a nucleic acid having a sequence that is more than about 20%, more than about 40%, more than about 60%, more than about 70%, more than about 75%, more than about 78%, more than about 80%, more than about 85%, more than about 90%, more than about 95%, more than about 97%, more than about 98%, or more than about 99% identical to SEQ ID NO: 1. In some embodiments, a nitrilase may be encoded by a nucleic acid comprising a nucleotide sequence that is more than about 90% identical to SEQ ID NO: 1 and/or may have a catalytic activity sufficient to convert at least about 50% of a nitrile to a carboxylic acid at a pH from about 5 to about 9 and a temperature less than 60° C.

In some embodiments, a nucleic acid encoding a nitrilase may be designed by modifying SEQ ID NO: 1 to optimize codon usage in an organism (e.g., a desired host organism). For example, Bradyrhizobium codons that are rare, inefficient, and/or error-prone in a desired host organism (e.g., E. coli) may be replaced with codons more suited to the host. A codon optimized nitrilase b116402 sequence may include, for example, SEQ ID NO: 5, which is about 78% identical to SEQ ID NO: 1.

A nucleic acid encoding a nitrilase, in some embodiments, may be designed by modifying SEQ ID NO: 1 to reduce or eliminate sequences that may adversely affect a desired expression level in a host organism. For example, if SEQ ID NO: 1 includes one or more sequences that may operate as expression control sequences in a particular host, these sequences may be modified or removed, at least to the extent that the modified sequence still encodes a nitrilase with a desired catalytic activity.

### EXAMPLES

Some embodiments of the disclosure may be illustrated by one or more of the following examples.

### Example 1

### Reduction of β-ketonitriles

An example procedure for the reduction of the β-ketonitriles is as follows: D-glucose (1.0 g), D-glucose dehydrogenase (10 mg), NADPH (10 mg) and ketoreductase (10 mg) were mixed in a potassium phosphate buffer (50 ml, 100 mM, pH 6.5). Into the mixture was added a ketonitrile solution (500 mg in 2.0 ml DMSO). The mixture was stirred at room temperature and pH was controlled at 6.5-6.6 with addition of 0.5 M NaOH solution until conversion was complete (usually overnight). The mixture was extracted with methyl tert-butyl ether. The organic extract was dried over anhydrous sodium sulfate and removal of the solvent gave product β-hydroxynitriles.

### Example 2

### Reduction of α-chloroketones

An example procedure for the reduction of the α-chloroketones is as follows: D-glucose (1.0 g), D-glucose dehydrogenase (10 mg), NADPH (10 mg) and ketoreductase (10 mg) were mixed in a potassium phosphate buffer (50 ml, 100 mM, pH 6.5). Into the mixture was added an αchloroketones solution (500 mg in 2.0 ml DMSO). The mixture was stirred at room temperature and pH was controlled at 6.5-6.6 with addition of 0.5 M NaOH solution until conversion was complete (usually overnight). The mixture was extracted with methyl tert-butyl ether. The organic extract was dried over anhydrous sodium sulfate and removal of the solvent gave product β-hydroxynitriles.

### Example 3

### Cyanization of α-chloroalcohols

An example procedure for the cyanization of α-chloroalcohols is as follows: (S)-2-chloro-1-(3,4-dichlorophenyl)ethanol (96 mg, 0.42 mmol) was dissolved in ethanol (10 ml). A solution of sodium cyanide (62 mg, 1.2 mmol) in water (5 ml) was added dropwise to the above solution during a period of 30 min. The reaction mixture was stirred at room temperature and monitored by TLC. Upon completion, the reaction mixture was acidified with 2N HCl solution to pH-5. The ethanol was removed and the remaining mixture was extracted with ethyl acetate. The extract was washed with brine and dried over anhydrous sodium sulfate. Removal of the solvent gave the crude product, which was purified by preparative TLC to give 3-(3,4-dichlorophenyl)-3-hydroxypropionitrile (61 mg, 66% yield).

### Example 4

### Hydrolysis of β-hydroxynitriles

An example procedure for the hydrolysis of β-hydroxynitriles is as follows: (S)-3-Hydroxy-3-phenylpropionitrile (120 mg, 0.8 mol) was dissolved in 40 ml of potassium phosphate buffer (100 mM, pH 7.2). Into the solution, 7 mg of nitrilase b116402 was added. The resulting mixture was stirred at 30° C., and the reaction was monitored with TLC. Upon completion, the reaction mixture was acidified with 2N HCl solution to pH being 4 to 5, and saturated with NaCl. The mixture was extracted with ethyl acetate, and the extract was dried with anhydrous sodium sulfate and solvent removed. Removal of the solvent gave the crude product, which was purified by preparative TLC to give (S)-3-hydroxy-3-phenylpropionic acid (120 mg, 89% yield).

Although the present invention and its advantages have been described in detail, it should be understood that various changes, substitutions and alternations can be made herein without departing from the spirit and scope of the invention as illustrated, in part, by the following claims.

What is claimed is:

1. A method of forming a composition comprising an enantiomeric excess of a β-hydroxy carboxylic acid, said method comprising:
contacting an α-haloketone with sodium cyanide in a composition to produce a β-ketonitrile;
contacting the β-ketonitrile with one or more ketoreductases under conditions that permit reduction of the β-ketonitrile to form a β-hydroxynitrile; and
contacting the β-hydroxynitrile with one or more nitrilases under conditions that permit hydrolysis of the β-hydroxynitrile to form an enantiomeric excess of an enantiomer of a β-hydroxy carboxylic acid in the composition.

2. A method according to claim 1, wherein the enantiomeric excess is over about 80 mole percent.

3. A method according to claim 1, wherein the enantiomeric excess is over about 95 mole percent.

4. A method according to claim 1, wherein the conditions that permit reduction of the β-ketonitrile to form a β-hydroxynitrile comprise a pH of between about 4 and about 10 and a temperature of less than about 60° C.

5. A method according to claim 1, wherein the conditions that permit hydrolysis of the β-hydroxynitrile comprise a pH of between about 4 and about 10 and a temperature of less than about 60° C.

6. A method according to claim 1, wherein the composition comprises water.

7. A method according to claim 1, wherein the composition comprises an organic solvent.

8. A method according to claim 1, wherein the α-haloketone is selected from the group consisting of 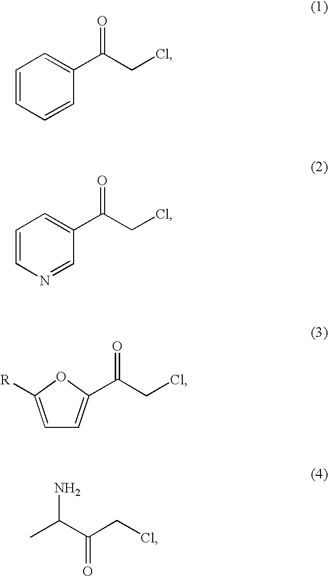
and variants thereof.

9. A method according to claim 1, wherein the β-hydroxy carboxylic acid is selected from the group consisting of 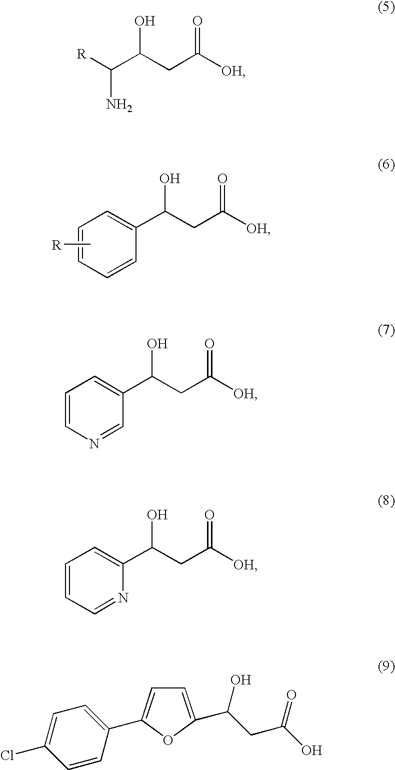
and variants thereof.

10. A method according to claim 1, wherein conditions that permit reduction of the β-ketonitrile further comprise contacting the β-ketonitrile and/or the ketoreductase with a cofactor.

11. A method according to claim 10, wherein the conditions that permit reduction of the β-ketonitrile further comprise contacting the cofactor with (a) glucose and glucose dehydrogenase, (b) formate and formate dehydrogenase, (c) glucose-6-phosphase and phosphate dehydrogenase, (d) molecular hydrogen and hydrogenase, (e) ethanol and alcohol dehydrogenase, or combinations of two or more of (a), (b), (c), and (d).

12. A method according to claim 10, wherein the cofactor comprises a molecule selected from the group consisting of NAD+, NADH, NADP+, and NADPH.

13. A method of forming a composition comprising an enantiomeric excess of a β-hydroxy carboxylic amide, said method comprising:
contacting an α-haloketone with sodium cyanide in a composition to produce a β-ketonitrile;
contacting the β-ketonitrile with one or more ketoreductases under conditions that permit reduction of the β-ketonitrile to form a β-hydroxynitrile; and
contacting the β-hydroxynitrile with one or more nitrile hydratases under conditions that permit hydrolysis of the β-hydroxynitrile to form an enantiomeric excess of an enantiomer of a β-hydroxy carboxylic amide in the composition.

14. A method according to claim 13 wherein the enantiomeric excess is over about 80 mole percent.

15. A method according to claim 13 wherein the enantiomeric excess is over about 95 mole percent.

16. A method according to claim 13, wherein the conditions that permit reduction of the β-ketonitrile to form a β-hydroxynitrile comprise a pH of between about 4 and about 10 and a temperature of less than about 60° C.

17. A method according to claim 13 wherein the conditions that permit hydrolysis of the β-hydroxynitrile comprise a pH of between about 4 and about 10 and a temperature of less than about 60° C.

18. A method according to claim 13 wherein the composition comprises water.

19. A method according to claim 13 wherein the composition comprises an organic solvent.

20. A method according to claim 13, wherein the α-haloketone is selected from the group consisting of 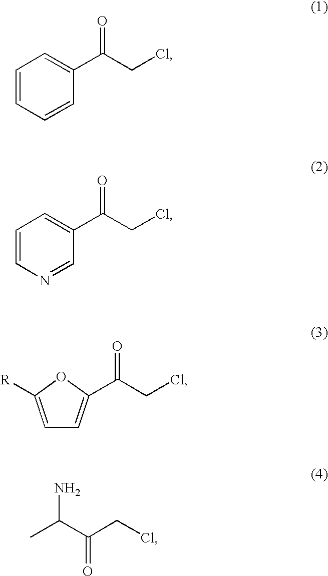
and variants thereof.

21. A method according to claim 13 wherein the β-hydroxycarboxylic amide is selected from the group consisting of 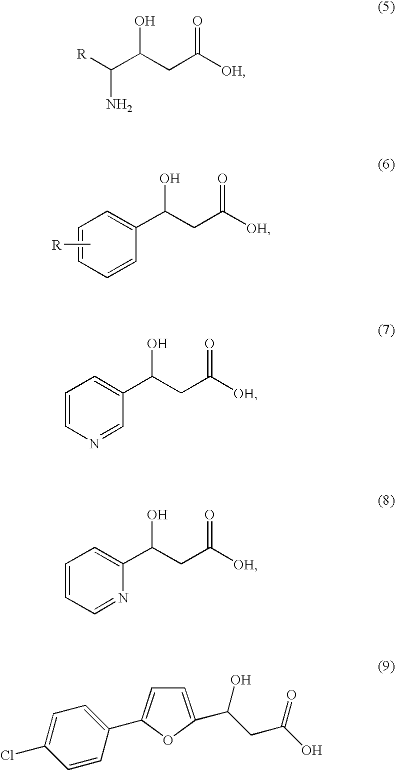
and variants thereof.

22. A method according to claim 13, wherein conditions that permit reduction of the β-ketonitrile further comprise contacting the β-ketonitrile and/or the ketoreductase with a cofactor.

23. A method according to claim 22, wherein the conditions that permit reduction of the β-ketonitrile further comprise contacting the cofactor with (a) glucose and glucose dehydrogenase, (b) formate and formate dehydrogenase, (c) glucose-6-phosphase and phosphate dehydrogenase, (d) molecular hydrogen and hydrogenase, (e) ethanol and alcohol dehydrogenase, or combinations of two or more of (a), (b), (c), and (d).

24. A method according to claim 22, wherein the cofactor comprises a molecule selected from the group consisting of NAD+, NADH, NADP+, and NADPH.

25. A method of forming a composition comprising an enantiomeric excess of a β-hydroxy carboxylic acid, said method comprising:
contacting an α-haloketone with one or more ketoreductases under conditions that permit reduction of the α-haloketone to produce an α-haloalcohol;
contacting the α-haloalcohol with sodium cyanide to form a β-hydroxynitrile; and
contacting the β-hydroxynitrile with one or more nitrilases under conditions that permit hydrolysis of the β-hydroxynitrile to form an enantiomeric excess of an enantiomer of a β-hydroxy carboxylic acid in the composition.

26. A method according to claim 25 wherein the enantiomeric excess is over about 80 mole percent.

27. A method according to claim 25 wherein the conditions that permit hydrolysis of the β-hydroxynitrile comprise a pH of between about 4 and about 10 and a temperature of less than about 60° C.

28. A method according to claim 25 wherein the α-haloketone is selected from the group consisting of 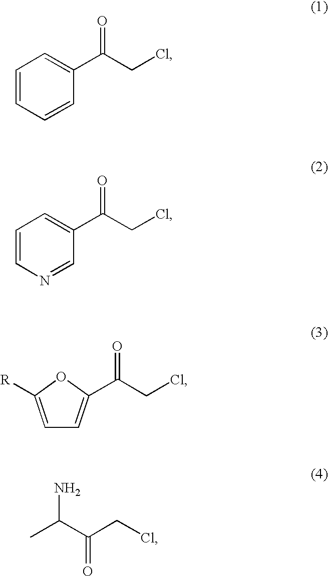
and variants thereof.

29. A method according to claim 25 wherein the β-hydroxycarboxylic acid is selected from the group consisting of 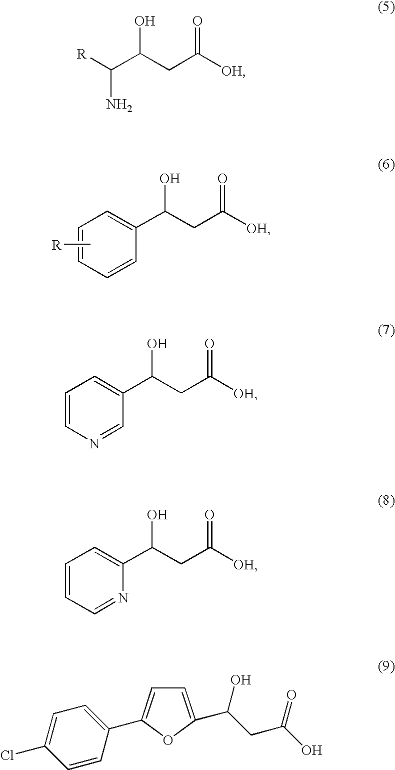
and variants thereof.

30. A method of forming a composition comprising an enantiomeric excess of a β-hydroxy carboxylic amide, said method comprising:
contacting an α-haloketone with one or more ketoreductases under conditions that permit reduction of the α-haloketone to produce an α-haloalcohol;
contacting the α-haloalcohol with sodium cyanide to form β-hydroxynitrile; and
contacting the β-hydroxynitrile with one or more nitrile hydratases under conditions that permit hydrolysis of the β-hydroxynitrile to form an enantiomeric excess of an enantiomer of a β-hydroxy carboxylic amide in the composition.

31. A method according to claim 30 wherein the enantiomeric excess is over about 80 mole percent.

32. A method according to claim 30 wherein the conditions that permit hydrolysis of the β-hydroxynitrile comprise a pH of between about 4 and about 10 and a temperature of less than about 60° C.

33. A method according to claim 30 wherein the α-haloketone is selected from the group consisting of 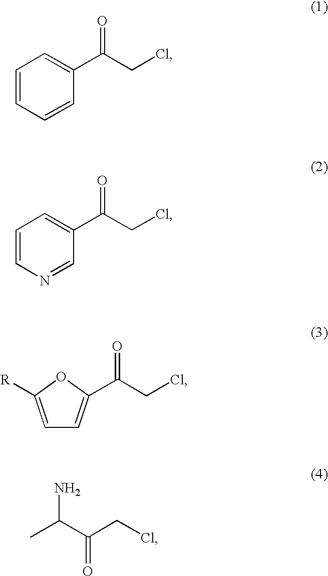
and variants thereof.

34. A method according to claim 30 wherein the β-hydroxy carboxylic amide is selected from the group consisting of 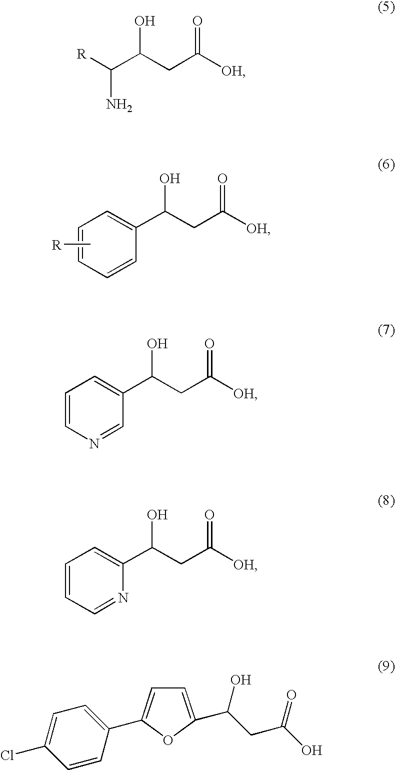
and variants thereof.

35. A method of forming a composition comprising an enantiomeric excess of a β-hydroxy carboxylic acid, said method comprising:
contacting an α-haloketone with sodium cyanide to produce a β-ketonitrile;
contacting the β-ketonitrile with one or more nitrilases under conditions that permit hydrolysis of the β-ketonitrile to form at least one β-keto acid; and
contacting the β-keto acid with a ketoreductase to form an enantiomeric excess of an enantiomer of a β-hydroxy carboxylic acid in the composition.

36. A method of forming a composition comprising an enantiomeric excess of a β-hydroxy carboxylic amide, said method comprising:
contacting an α-haloketone with sodium cyanide to produce a β-ketonitrile;
contacting the β-ketonitrile with one or more nitrile hydratases under conditions that permit hydrolysis of the β-ketonitrile to form at least one β-keto amide; and
contacting the β-keto amide with a ketoreductase to form to form an enantiomeric excess of an enantiomer of a β-hydroxy carboxylic amide in the composition.


SEQUENCE LISTING (5 sequences)

SEQ ID NO: 1 - DNA, 1005 bp, Bradyrhizobium japonicum
atgcaggacacgaaattcaaagtcgcggtcgttcaggctgcgccggtattcatggatgcgccagcttccgtggccaaggcgattggtttcattgcggaggcgggtgcagcgggggcgaagctgctggcgttcccggaggtctggattccgggctatccttggtggctttggctcgggacgccggcttggggaatgcagtttgtaccccgctatcacgccaattcgctgcgtgctgatgggcccgacatcctcgcactgtgtgcggccgccgccgaagcgaaaatcaatgtggtgatgggcttctccgaaatcgacggaggaacgctctacctaagtcaggtctttattagcgatgcgggagagatcatcttcaagcgccgaaagctcaagccgacacacgtcgaacgtacgctctatggcgaaggagatgggtctgatttccgcgtcgtcgaaagcagcgtcggacgtctcggagccttgtgctgcgccgagcacattcagccgttgtcgaaatacgccatgtactcgatgaacgagcaagttcacgtggcgtcgtggccatcttttacgctgtatcgcgacaaggcctacgctttgggccatgaggtgaatctcgccgccagccagatctacgcgctagagggaggttgcttcgtcctgcatgcctcggcaattactggtcaagatatgttcgacatgctttgcgatactccggaaaaggccgatttgctgaacgcggagggagcgaagccgggtggaggctattcgatgatcttcggtcccgatggtcagccgatgtgcgagcatctgccgcaggacaaggaaggcatcctctatgctgacgtggacctgtcaatgatcgcaatcgccaaagcggcctacgaccccacggggcattacgcccgcggcgatgtcgtccgtctcatggtcaatcgcagcccgcgtcgcacgagcgtcagcttcagcgaagacgagaacgcggcggtcactttcaccgagacttga

SEQ ID NO: 2 - protein, 334 aa, Bradyrhizobium japonicum
MQDTKFKVAVVQAAPVFMDAPASVAKAIGFIAEAGAAGAKLLAFPEVWIPGYPWWLWLGTPAWGMQFVPRYHANSLRADGPDILALCAAAAEAKINVVMGFSEIDGGTLYLSQVFISDAGEIIFKRRKLKPTHVERTLYGEGDGSDFRVVESSVGRLGALCCAEHIQPLSKYAMYSMNEQVHVASWPSFTLYRDKAYALGHEVNLAASQIYALEGGCFVLHASAITGQDMFDMLCDTPEKADLLNAEGAKPGGGYSMIFGPDGQPMCEHLPQDKEGILYADVDLSMIAIAKAAYDPTGHYARGDVVRLMVNRSPRRTSVSFSEDENAAVTFTET

SEQ ID NO: 3 - DNA, 31 bp, Artificial Sequence
  Description of artificial sequence Bradyrhizobium japonicum USDA110 bll6402 forward PCR primer
tcgcatatgcaggacacgaaattcaaagtcg

SEQ ID NO: 4 - DNA, 28 bp, Artificial Sequence
  Description of artificial sequence Bradyrhizobium japonicum USDA110 bll6402 reverse PCR primer
aaactcgagagtctcggtgaaagtgacc

SEQ ID NO: 5 - DNA, 1002 bp, Artificial Sequence
  Description of artificial sequence b116402 optimized for expression in E. coli using DNA star
atgcaggacaccaaattcaaagttgctgttgttcaggctgctccggttttcatggacgctccggcttctgttgctaaagctatcggtttcatcgctgaagctggtgctgctggtgctaaactgctggctttcccggaagtttggatcccgggttacccgtggtggctgtggctgggtaccccggcttggggtatgcagttcgttccgcgttaccacgctaactctctgcgtgctgacggtccggacatcctggctctgtgcgctgctgctgctgaagctaaaatcaacgttgttatgggtttctctgaaatcgacggtggtaccctgtacctgtctcaggttttcatctctgacgctggtgaaatcatcttcaaacgtcgtaaactgaaaccgacccacgttgaacgtaccctgtacggtgaaggtgacggttctgacttccgtgttgttgaatcttctgttggtcgtctgggtgctctgtgctgcgctgaacacatccagccgctgtctaaatacgctatgtactctatgaacgaacaggttcacgttgcttcttggccgtctttcaccctgtaccgtgacaaagcttacgctctgggtcacgaagttaacctggctgcttctcagatctacgctctggaaggtggttgcttcgttctgcacgcttctgctatcaccggtcaggacatgttcgacatgctgtgcgacaccccggaaaaagctgacctgctgaacgctgaaggtgctaaaccgggtggtggttactctatgatcttcggtccggacggtcagccgatgtgcgaacacctgccgcaggacaaagaaggtatcctgtacgctgacgttgacctgtctatgatcgctatcgctaaagctgcttacgacccgaccggtcactacgctcgtggtgacgttgttcgtctgatggttaaccgttctccgcgtcgtacctctgtttctttctctgaagacgaaaacgctgctgttaccttcaccgaaacc